(19) United States
(12) Patent Application Publication
(10) Pub. No.: US 2026/0269020 A1
(43) Pub. Date: Sep. 10, 2026

(54) STRATEGY FOR EFFICIENT SOLVENT CONDITION PREDICTION

(71) Applicant: BASF SE, Ludwigshafen am Rhein (DE)

(72) Inventors: Frieda Anna SORGENFREI, Graz (AT); Wolfgang KROUTIL, Graz (AT); Stefan SEEMAYER, Ludwigshafen am Rhein (DE); Florian WEISSENSTEINER, Graz (AT); Marco ZECHNER, Graz (AT)

(21) Appl. No.: 19/168,033

(22) PCT Filed: Mar. 28, 2024
(86) PCT No.: PCT/EP2024/058635

§ 371 Date: Sep. 23, 2025

(30) Foreign Application Priority Data
Mar. 29, 2023 (EP) 23165017.7

(51) Int. Cl. G16C 20/10; G16C 20/50

(52) U.S. Cl. CPC G16C 20/10; G16C 20/50

(57) ABSTRACT

Described herein is a method for providing three reaction parameters for an enzymatic reaction depending on another reaction parameter, where the parameters are selected from a particular enzyme, a reaction temperature, a solvent, and the solvent concentration. Also described herein are a computer program for providing three reaction parameters for an enzymatic reaction depending on another reaction parameter, a non-transitory computer readable data medium and a system for providing three reaction parameters for an enzymatic reaction depending on another reaction parameter.

The present invention relates to a method for providing three reaction parameters for an enzymatic reaction depending on another reaction parameter, wherein the parameters are selected from a particular enzyme, a reaction temperature, a solvent, and the solvent concentration. The present invention further relates to a computer program for providing three reaction parameters for an enzymatic reaction depending on another reaction parameter, to a non-transitory computer readable data medium and to a system for providing three reaction parameters for an enzymatic reaction depending on another reaction parameter.

The activity of an enzyme, and thus the space-time-yield of an enzymatic reaction, is influenced by several reaction parameters. To provide a possibly high yield of an enzymatic reaction, or to ensure a highly active enzyme, such reactions are typically optimized before larger scale reactions are conducted or before higher amounts of a limited substrate are used.

Enzymatic reactions are often supplemented with a solvent, to obtain the possibility to optimize the reaction properties and bypass a variety of important challenges. However, it was found that solvents have an impact on the activity of enzymes. Thus, for optimization of an enzymatic reaction, not only the selection of the enzyme and the reaction temperature, but also the solvent and its concentration are important parameters to be considered.

However, optimizing an enzymatic reaction including a solvent or a solvent mixture remains challenging and requires high efforts.

Currently, two approaches exist for optimizing an enzymatic reaction.

One approach includes measuring the enzyme activity in every sample to find the best reaction conditions which, however, is very time intense and consumes high amounts of resources including enzymes and substrate. Particularly for large screenings, it can become unfeasible if the reaction cannot be coupled to a simple read-out like the shift of pH (e.g. as described in Bollinger, A. et al. “Organic-solvent-tolerant carboxylic ester hydrolases for organic synthesis”, Appl. Environ. Microbiol. 86, (2020)) but is dependent on measuring the transformation of a substrate in every sample for finding optimal reaction conditions.

An alternative to this tedious approach is the assessment of enzyme stability by means of the melting temperature, as described in Kazlauskas, R. “Engineering more stable proteins” Chem. Soc. Rev. 47, 9026-9045 (2018). —Chapter 3. Measuring the folding-unfolding equilibrium. The melting temperature is defined as the temperature at which equal amounts of the enzyme are folded and unfolded under the given conditions.

Different methods to determine the melting temperature have been described, which share the principle that a sample is gradually heated and the enzyme changes from a folded to an unfolded state. This process can be monitored for example (i) by measuring the fluorescence signal of internal tryptophane residues, (ii) by using a fluorescent dye creating a change in fluorescence upon interaction with the unfolding protein or (iii) in the case of ene reductases following the change of fluorescence of the cofactor FMN.

For example, Arroyo et al., “Prediction Of Penicillin V Acylase Stability In Water-Organic Co-Solvent Monophasic Systems As A Function Of Solvent Composition”, ENZYME AND MICROBIAL TECHNOLOGY, vol. 27, 1 Jul. 2000 (2000-07-01), pages 122-126 describes methods for determining protein denaturation characteristics by observing a decline in enzymatic activity as solvent concentrations increase. However, these methods necessitate extensive measurements to establish maximum activity and the rate of activity loss for each solvent concentration. Additionally, these measurements must be repeated for each desired temperature. Another drawback is that determining enzymatic activity requires substantial experimental efforts, such as observing substrate turnover rates, providing ample co-factors, and preventing product inhibition by removing reaction products. As will be described below, the present invention introduces a highly efficient approach for determining various protein characteristics based on a limited number of measurements.

Many examples of enzymes with elevated thermal stability have been described which were identified either from extremophiles possessing, in general, an inherently elevated tolerance to temperature or by enzyme engineering.

In recent years, the strategies and methods in protein engineering have moved towards data driven rational or semi-rational design. A number of computational tools and strategies have been published either to predict thermal stability or to suggest and design variants with improved thermal properties. The computational “Framework for Rapid Enzyme Stabilization by Computational libraries” (FRESCO) was successfully employed on different enzyme classes, covering hydrolases, Bayer-Villinger monooxygenases, alcohol dehydrogenases and transaminases to optimize their thermal properties. Besides this, other strategies including ancestral sequence reconstruction, sequence and structure and molecular dynamics guided design yielded enzymes with increased thermal resistance. There are also examples of improving the thermal tolerance of ene reductases e.g. by rational design. In general, a raised melting point or thermotolerance was described to go in hand with an increased tolerance to solvents in several of these examples.

Although the melting temperature is used as a basis for optimizing enzymatic reactions, even reactions including solvents or solvent mixtures, this parameter cannot serve as a quantitative measure for enzyme activity, particularly since optimizing a reaction based on the melting temperature does not consider the influence of a particular solvent and its concentration on the enzyme's activity. Furthermore, the approach based on the melting temperature does not supply a solvent concentration threshold up to which an active enzyme can be expected and thus provides only limited information for optimizing such reactions.

Consequently, alternative strategies are required to simplify assessing the solvent tolerance of enzymes. In selected cases, for example, it has already been reported that enzymes showed actually higher activity at low solvent concentration compared to reaction conditions without supplemented solvent. The reasons for this may be manifold. It is suggested that structural changes or increased mobility in the enzyme through modified hydrogen bond networks could be the reason. Nevertheless, no model has been deduced to describe and quantify this behaviour.

A primary object of the present invention was thus to find a possibility for determining suitable reaction parameters, such as the particular enzyme, the reaction temperature, the selected solvent and the concentration of the solvent, particularly wherein the approach is more precise and requires less time and resources than current approaches.

The above-mentioned primary object of the present invention is solved by a method, preferably computer-implemented method, for providing three reaction parameters (a), (b) and (c) for an enzymatic reaction depending on a reaction parameter (d), wherein the reaction parameters (a), (b), (c) and (d) are selected from the group consisting of a particular enzyme, a reaction temperature, a particular solvent, and the solvent concentration in an aqueous system,
wherein each parameter selected from the group consisting of a particular enzyme, a reaction temperature, a solvent, and the solvent concentration is selected as one of the reaction parameters (a), (b), (c) and (d),
the method comprising the steps of:
i) providing or receiving measurement data representing an evolution of the ratio of folded to unfolded enzyme in dependence on a variation of one or more solvent(s) and the solvent concentration(s) of the of one or more solvent(s) over a predefined temperature range, for one or more enzyme(s),
ii) determining from the evolution of the ratio of folded to unfolded enzyme a concentration of the solvent for half-unfolding of the enzyme for selected temperatures within the predefined temperature range,
iii) based on the determination of step ii), providing one or more suitable
   reaction temperature(s),
   solvent(s),
   solvent concentration(s), and
   optionally: enzyme(s)
for use in the enzymatic reaction, wherein suitable refers to obtaining a ratio of folded to unfolded enzyme of at least 1:1.

It was found that approaches for optimizing reaction conditions, which are based on the melting temperature, sometimes provide adequate results. However, in other cases, the results are not suitable (see Example 1). Thus, it was found that the melting temperature is not a suitable parameter for optimizing conditions for reactions including a solvent.

However, it was surprisingly found that approaches based on the solvent concentration c_{U50}, i.e., the solvent concentration at which half of the respective enzyme is folded, advantageously match with the measured enzymatic activity and are thus suitable to provide predictions regarding enzyme activity (see Example 3). Thus, with a method according to the invention, it is possible to provide a prediction with which reaction parameters an active enzyme is obtained.

Surprisingly, the inventors have discovered that by directly observing protein unfolding to identify the conditions for half-maximal unfolding, only a minimal number of measurements (sometimes as few as 2) are necessary to predict protein activities under various conditions. This approach eliminates the need to determine optimal enzymatic activity conditions and instead allows for predictions based on measurements of protein unfolding and the identification of the conditions for half-maximal unfolding.

In some prior art documents such as
Padua et al., “ThermoFMN—A Thermofluor Assay Developed for Ligand-Screening as an Alternative Strategy for Drug Discovery”, JOURNAL OF THE BRAZILIAN CHEMICAL SOCIETY, 1 Jan. 2014,
Kazlauskas, “Engineering more stable proteins”, CHEMICAL SOCIETY REVIEWS, vol. 47, no. 24, 1 Jan. 2018 (2018-01-01), pages 9026-9045,
Hung Chien-Lun et al., “Protein Stability Depends Critically on the Surface Hydrogen-Bonding Network: A Case Study of Bid Protein”, JOURNAL OF PHYSICAL CHEMISTRY PART B, vol. 125, no. 30, 27 Jul. 2021 (2021-07-27), pages 8373-8382, or
Zwanzig, “Two-state models of protein folding kinetics”, PROCEEDINGS OF THE NATIONAL ACADEMY OF SCIENCES, vol. 94, no. 1, 7 Jan. 1997 (1997-01-07), pages 148-150,
   a relationship between loss of enzymatic activity and denaturation is discussed. However, these documents only provide a generic understanding of this relationship. None of these documents discloses or suggest that precise predictions of enzymatic activities under diverse conditions are possible by simply measuring protein denaturation and determining the point of half-denaturation. To achieve such accurate predictions, it would have been necessary to suggest several suitable parameters simultaneously, such as temperature, enzyme, solvent, and solvent concentration. Therefore, merely performing a calibration as described by Arroyo et al. (as described above) would not provide a prediction of suitable reaction conditions for uncalibrated parameters.

The term “c_{U50}” is defined as the solvent concentration, at which equal amounts of folded and unfolded protein are present.

Preferably, the term “active enzyme” refers to a ratio of folded to unfolded enzyme of at least 1:1, as described herein.

The term “wherein each parameter selected from the group consisting of a particular enzyme, a reaction temperature, a solvent, and the solvent concentration is selected as one of the reaction parameters (a), (b), (c) and (d)” is to be understood such that each of the listed parameters is selected as one of reaction parameters (a) to (d), wherein each of the listed parameters is selected as only one of parameters (a) to (d).

The term “providing measurement data” in step i) may include generating the measurement data to be used in the further course of the method. For example, the measurement data may be generated by processing a sensor signal transmitted by a sensor during the measurement. Measurement data may also be generated by processing raw data obtained during a measurement. The term “receiving measurement data” may include that the measurement data to be used in the further course of the method are obtained from a memory or cloud storing the measurement data, e.g., in a database.

The term “measurement data representing an evolution of the ratio of folded to unfolded enzyme in dependence on a variation of one or more solvent(s) and the solvent concentration(s) of the one or more solvent(s) over a predefined temperature range” is to be understood such that the measurement data shows different ratios of folded to unfolded enzyme for different solvents and their concentrations and different temperatures, wherein the ratio is measured for temperatures in a predefined temperature range.

The term “one or more enzyme(s)” in step i) is to be understood as one, two, three or more enzymes. Preferably, the term is to be understood as at least one, preferably at least two, preferably at least three, preferably at least four, preferably at least five, preferably at least six, preferably at least seven, preferably at least eight, preferably at least nine, preferably at least ten, preferably at least twelve, preferably at least fifteen, preferably at least twenty, preferably at least twenty five, preferably at least thirty, preferably at least forty, preferably at least fifty enzymes.

Preferably, the, one, two, three or more or all enzyme(s) in step i) is/are selected from the group consisting of oxidoreductases, transferases, hydrolases, lyases, isomerases, ligases and translocases.

However, the method according to the invention is based on a general principle of enzymatic reactions and can thus be applied to any enzymatic reaction, with any enzyme.

Preferably, the, one, two, three or more or all enzyme(s) in step i) is/are selected from the group consisting of oxidoreductases that act on the CH—OH group of donors (EC 1.1), oxidoreductases that act on the aldehyde or oxo group of donors (EC 1.2), oxidoreductases that act on the CH—CH group of donors (EC 1.3), oxidoreductases that act on the CH—NH2 group of donors (Amino acid oxidoreductases, Monoamine oxidase) (EC 1.4), oxidoreductases that act on CH—NH group of donors (EC 1.5), oxidoreductases that act on NADH or NADPH (EC 1.6), oxidoreductases that act on other nitrogenous compounds as donors (EC 1.7), oxidoreductases that act on a sulfur group of donors (EC 1.8), oxidoreductases that act on a heme group of donors (EC 1.9), oxidoreductases that act on diphenols and related substances as donors (EC 1.10), oxidoreductases that act on peroxide as an acceptor (peroxidases) (EC 1.11), oxidoreductases that act on hydrogen as donors (EC 1.12), oxidoreductases that act on single donors with incorporation of molecular oxygen (oxygenases) (EC 1.13), oxidoreductases that act on paired donors with incorporation of molecular oxygen (EC 1.14), oxidoreductases that act on superoxide radicals as acceptors (EC 1.15), oxidoreductases that oxidize metal ions (EC 1.16), oxidoreductases that act on CH or CH2 groups (EC 1.17), oxidoreductases that act on iron-sulfur proteins as donors (EC 1.18), oxidoreductases that act on reduced flavodoxin as a donor (EC 1.19), oxidoreductases that act on phosphorus or arsenic in donors (EC 1.20), oxidoreductases that act on X—H and Y—H to form an X-Y bond (EC 1.21), Methylases (EC 2.1), transketolases (EC 2.2), transaldolases (EC 2.2), acyltransferases (EC 2.3), glycosyltransferases (EC 2.4), Transferases transferring alkyl or aryl groups, other than methyl groups (EC 2.5), Transferases Transferring nitrogenous groups (EC 2.6), Transferases transferring phosphorus-containing groups (EC 2.7), Transferases transferring Sulfur-Containing Groups (EC 2.8), L-seryl-tRNASec selenium transferase or O-phospho-L-seryl-tRNASec: L-selenocysteinyl-tRNA synthase (EC 2.9), hydrolases that act on ester bonds (esterases, nucleases, phosphodiesterases, lipase, phosphatase) (EC 3.1), hydrolases that act on sugars (DNA glycosylases, glycoside hydrolase) (EC 3.2), hydrolases that act on ether bonds (EC 3.3), hydrolases that act on peptide bonds (Proteases/peptidases) (EC 3.4), hydrolases that act on carbon-nitrogen bonds, other than peptide bonds (EC 3.5), hydrolases that act on acid anhydrides (acid anhydride hydrolases, including helicases and GTPase) (EC 3.6), hydrolases that act on carbon-carbon bonds (EC 3.7), hydrolases that act on halide bonds (EC 3.8), hydrolases that act on phosphorus-nitrogen bonds (EC 3.9), hydrolases that act on sulphur-nitrogen bonds (EC 3.10), hydrolases that act on carbon-phosphorus bonds (EC 3.11), hydrolases that act on sulfur-sulfur bonds (EC 3.12), hydrolases that act on carbon-sulfur bonds (EC 3.13), carbon-carbon lyases (EC 4.1), carbon-oxygen lyases (EC 4.2), carbon-nitrogen lyases (EC 4.3), carbon-sulfur lyases (EC 4.4), carbon-halide lyases (EC 4.5), phosphorus-oxygen lyases (EC 4.6), racemases and epimerases (EC 5.1), cis-trans-isomerases (EC 5.2), intramolecular oxidoreductases (EC 5.3), intramolecular transferases-mutases (EC 5.4), intramolecular lyases (EC 5.5), other isomerases (EC 5.99), ligases forming carbon-oxygen bonds (EC 6.1), ligases forming carbon-sulfur bonds (EC 6.2), ligases forming carbon-nitrogen bonds (including argininosuccinate synthe-tase) (EC 6.3), ligases forming carbon-carbon bonds (EC 6.4), ligases forming phosphoric ester bonds (EC 6.5), and ligases forming nitrogen-metal bonds (EC 6.6).

Preferably, the, one, two, three or more or all enzyme(s) in step i) is/are selected from the group consisting of oxidoreductases that act on the CH—OH group of donors (EC 1.1), oxidoreductases that act on the aldehyde or oxo group of donors (EC 1.2), oxidoreductases that act on the CH—CH group of donors (EC 1.3), oxidoreductases that act on the CH—NH₂ group of donors (Amino acid oxidoreductases, Monoamine oxidase) (EC 1.4), oxidoreductases that act on CH—NH group of donors (EC 1.5), oxidoreductases that act on NADH or NADPH (EC 1.6), oxidoreductases that act on other nitrogenous compounds as donors (EC 1.7), oxidoreductases that act on a sulfur group of donors (EC 1.8), oxidoreductases that act on a heme group of donors (EC 1.9), oxidoreductases that act on diphenols and related substances as donors (EC 1.10), oxidoreductases that act on peroxide as an acceptor (peroxidases) (EC 1.11), oxidoreductases that act on hydrogen as donors (EC 1.12), oxidoreductases that act on single donors with incorporation of molecular oxygen (oxygenases) (EC 1.13), oxidoreductases that act on paired donors with incorporation of molecular oxygen (EC 1.14), oxidoreductases that act on superoxide radicals as acceptors (EC 1.15), oxidoreductases that oxidize metal ions (EC 1.16), oxidoreductases that act on CH or CH2 groups (EC 1.17), oxidoreductases that act on iron-sulfur proteins as donors (EC 1.18), oxidoreductases that act on reduced flavodoxin as a donor (EC 1.19), oxidoreductases that act on phosphorus or arsenic in donors (EC 1.20), and oxidoreductases that act on X—H and Y—H to form an X-Y bond (EC 1.21).

Preferably, the, one, two, three or more or all enzyme(s) in step i) is/are selected from the group consisting of Methylases (EC 2.1), transketolases (EC 2.2), transaldolases (EC 2.2), acyltransferases (EC 2.3), glycosyltransferases (EC 2.4), Transferases transferring alkyl or aryl groups, other than methyl groups (EC 2.5), Transferases Transferring nitrogenous groups (EC 2.6), Transferases transferring phosphorus-containing groups (EC 2.7), Transferases transferring Sulfur-Containing Groups (EC 2.8), and L-seryl-tRNASec selenium transferase or O-phospho-L-seryl-tRNASec: L-selenocysteinyl-tRNA synthase (EC 2.9).

Preferably, the, one, two, three or more or all enzyme(s) in step i) is/are selected from the group consisting of hydrolases that act on ester bonds (esterases, nucleases, phosphodiesterases, lipase, phosphatase) (EC 3.1), hydrolases that act on sugars (DNA glycosylases, glycoside hydrolase) (EC 3.2), hydrolases that act on ether bonds (EC 3.3), hydrolases that act on peptide bonds (Proteases/peptidases) (EC 3.4), hydrolases that act on carbon-nitrogen bonds, other than peptide bonds (EC 3.5), hydrolases that act on acid anhydrides (acid anhydride hydrolases, including helicases and GTPase) (EC 3.6), hydrolases that act on carbon-carbon bonds (EC 3.7), hydrolases that act on halide bonds (EC 3.8), hydrolas-es that act on phosphorus-nitrogen bonds (EC 3.9), hydrolases that act on sulphur-nitrogen bonds (EC 3.10), hydrolases that act on carbon-phosphorus bonds (EC 3.11), hydrolases that act on sulfur-sulfur bonds (EC 3.12), and hydrolases that act on carbon-sulfur bonds (EC 3.13).

Preferably, the, one, two, three or more or all enzyme(s) in step i) is/are selected from the group consisting of carbon-carbon lyases (EC 4.1), carbon-oxygen lyases (EC 4.2), carbon-nitrogen lyases (EC 4.3), carbon-sulfur lyases (EC 4.4), carbon-halide lyases (EC 4.5), and phosphorus-oxygen lyases (EC 4.6).

Preferably, the, one, two, three or more or all enzyme(s) in step i) is/are selected from the group consisting of racemases and epimerases (EC 5.1), cis-trans-isomerases (EC 5.2), intramolecular oxidoreductases (EC 5.3), intramolecular transferases-mutases (EC 5.4), intramolecular lyases (EC 5.5), and other isomerases (EC 5.99).

Preferably, the, one, two, three or more or all enzyme(s) in step i) is/are selected from the group consisting of ligases forming carbon-oxygen bonds (EC 6.1), ligases forming carbon-sulfur bonds (EC 6.2), ligases forming carbon-nitrogen bonds (including argininosuccinate synthetase) (EC 6.3), ligases forming carbon-carbon bonds (EC 6.4), ligases forming phosphoric ester bonds (EC 6.5), and ligases forming nitrogen-metal bonds (EC 6.6).

The EC numbers described above refer to the classification in the International Union of Biochemistry and Molecular Biology's Enzyme Commission (EC) numbering system.

The term “one or more solvent(s)” in step i) is to be understood as one, two, three or more solvents. Preferably, the term is to be understood as at least one, preferably at least two, preferably at least three, preferably at least four, preferably at least five, preferably at least six, preferably at least seven, preferably at least eight, preferably at least nine, preferably at least ten solvents.

Preferably, the, one, two, three or more or all solvent(s) in step i) is/are selected from the group consisting of aliphatic or cycloaliphatic or aromatic hydrocarbons and their halogen or nitro derivatives, their alcohols, their glycols, their ethers, their ketones, their carboxylic acids, their esters, their amides, their nitriles, their amines, their sulfoxides, and their heteroarenes; deep eutectic solvents, preferably natural deep eutectic solvents; CO₂, preferably supercritical CO₂; and ionic liquids.

Preferably, the, one, two, three or more or all solvent(s) in step i) is/are selected from the group consisting of pentane, hexane, benzene, heptane, toluene, 1,4-dioxane, diethyl ether, tetrahydrofuran (THF), Chloroform, Dichloromethane (DCM), ethyl acetate, acetone, dimethylformamide (DMF), acetonitrile (MeCN), dimethyl sulfoxide (DMSO), nitromethane, propylene carbonate, ammonia, formic acid, butanol, isopropyl alcohol (IPA), propanol (preferably isopropanol; preferably 1-propanol), ethanol, methanol, acetic acid, water, deep eutectic solvents (preferably natural deep eutectic solvents), CO₂ (preferably supercritical CO₂) and ionic liquids.

Preferably, the term “ionic liquid” refers to a liquid composition comprising or consisting of a salt in a liquid state. Preferably, the salt has a melting point of at most 100° C.

Preferably, the method according to the invention further comprises the step
I measuring an evolution of the ratio of folded to unfolded enzyme to obtain measurement data for step i).

It is preferred that step i) comprises the steps of
i.a) providing one or more enzyme(s),
i.b) providing one or more solvent(s),
i.c) mixing the enzyme(s) provided in step i) with the solvent(s) provided in step ii) to obtain a plurality of samples,
   wherein each sample comprises one of the enzyme(s) provided in step i) and one of the solvent(s) provided in step ii) in a predetermined solvent concentration,
   wherein for each enzyme provided in step i.a) at least five samples are obtained for each solvent provided in step i.b) and mixed with the enzyme, wherein the predetermined solvent concentration is different between the at least five samples,
i.d) measuring in each of the samples obtained in step i.e) the ratio of folded to unfolded enzyme over a predefined temperature range, and
i.e) generating measurement data representing the measured ratio of folded to unfolded enzyme over a predefined temperature range in each of the samples.

For example, enzymes A and B are provided in step i.a) and solvents X and Y are provided in step i.b). Thus, the mixing step in step i.e) generally provides the samples A+X, A+Y, B+X, and B+Y, wherein for each of the combinations A+X, A+Y, B+X, and B+Y, the solvent X or, respectively, Y is present in five different concentrations (e.g. X1, X2, X3, X4, and X5). Thus, the plurality of samples includes A+X1, A+X2, A+X3, A+X4, A+X5, A+Y1, A+Y2, A+Y3, A+Y4, A+Y5, B+X1, B+X2, B+X3, B+X4, B+X5, B+Y1, B+Y2, B+Y3, B+Y4, and B+Y5. The same applies accordingly for different numbers of provided enzyme(s), solvent(s) and solvent concentrations. For each of these samples, the ratio of folded to unfolded enzyme is measured over a predefined temperature range.

Preferably, the term “measured over a predefined temperature range” does not require a continuous measurement, but may refer to at least two, preferably at least three, preferably at least four, preferably at least five, preferably at least six, preferably at least seven, preferably at least eight, preferably at least nine, preferably at least ten, preferably at least twelve, preferably at least fifteen, preferably at least twenty, preferably at least twenty five, preferably at least thirty, preferably at least forty, preferably at least fifty, preferably at least hundred, preferably at least two hundred, preferably at least three hundred, preferably at least four hundred, preferably at least five hundred, preferably at least six hundred, preferably at least seven hundred different temperatures within a predefined temperature range.

The term “the predetermined solvent concentration is different between the at least five samples” is to be understood that each of the at least five samples has a solvent concentration, which is different from the other samples of the at least five samples.

In case in step i.a) more than one enzyme is provided, the at least five samples obtained in step i.e) are obtained for each of the provided enzymes. In this case, it is preferred that the at least five samples obtained for one enzyme correspond to the at least five samples obtained for the or all other enzyme(s) and thus only differ in the presence of another enzyme.

For example, enzymes A and B are provided in step i.a) and solvent X is provided in step i.b). Thus, the mixing step in step i.e) generally provides the samples A+X, and B+X, wherein for each of the combinations A+X and B+X, the solvent X is present in five different concentrations (e.g. X1, X2, X3, X4, and X5). Thus, the plurality of samples includes A+X1, A+X2, A+X3, A+X4, A+X5, B+X1, B+X2, B+X3, B+X4, and B+X5, wherein preferably the solvent concentration X1 in A+X1 corresponds to the solvent concentration X1 in B+X1. The same applies accordingly for concentrations X2, X3, X4, and X5.

In case in step i.b) more than one solvent is provided, the at least five samples obtained in step i.e) are obtained for each of the provided solvents. In this case, it is preferred that the at least five samples obtained for one solvent correspond to the at least five samples obtained for the or all other solvent(s) and thus only differ in the presence of another solvent.

For example, enzyme A is provided in step i.a) and solvents X and Y are provided in step i.b). Thus, the mixing step in step i.e) generally provides the samples A+X, and A+Y, wherein for each of the combinations A+X and A+Y, the solvent X or, respectively, Y is present in five different concentrations (e.g. X1, X2, X3, X4, and X5). Thus, the plurality of samples includes A+X1, A+X2, A+X3, A+X4, A+X5, A+Y1, A+Y2, A+Y3, A+Y4 and A+Y5, wherein preferably the solvent concentration X1 in A+X1 corresponds to the solvent concentration Y1 in A+Y1. The same applies accordingly for concentrations X2, X3, X4, and X5.

In case in step i.a) more than one enzyme is provided and in step i.b) more than one solvent is provided, the at least five samples obtained in step i.e) for each enzyme are obtained for each of the provided solvents. In this case, it is preferred that the at least five samples obtained for one enzyme and one solvent correspond to the at least five samples obtained for the or all other enzyme(s) and/or the or all other solvent(s).

For example, enzymes A and B are provided in step i.a) and solvents X and Y are provided in step i.b). Thus, the mixing step in step i.e) generally provides the samples A+X, A+Y, B+X, and B+Y, wherein for each of the combinations A+X, A+Y, B+X, and B+Y, the solvent X or, respectively, Y is present in five different concentrations (e.g. X1, X2, X3, X4, and X5). Thus, the plurality of samples includes A+X1, A+X2, A+X3, A+X4, A+X5, A+Y1, A+Y2, A+Y3, A+Y4, A+Y5, B+X1, B+X2, B+X3, B+X4, B+X5, B+Y1, B+Y2, B+Y3, B+Y4, and B+Y5, wherein preferably the solvent concentration X1 in A+X1 corresponds to the solvent concentration X1 in B+X1, and/or to the solvent concentration Y1 in A+Y1 and/or to the solvent concentration Y1 in B+Y1. Preferably, the solvent concentration X1 in A+X1 corresponds to the solvent concentration X1 in B+X1, and to the solvent concentration Y1 in A+Y1 and to the solvent concentration Y1 in B+Y1. The same applies accordingly for concentrations X2, X3, X4, and X5.

It is preferred that in step i.e) for each enzyme provided in step i.a) at least six, preferably at least seven, preferably at least eight, preferably at least nine, preferably at least ten, preferably at least twelve, preferably at least fifteen, preferably at least twenty, preferably at least twenty five, preferably at least thirty, preferably at least forty, preferably at least fifty samples are obtained for each solvent provided in step i.b) and mixed with the enzyme,
wherein the predetermined solvent concentration is different between the, at least six, at least seven, at least eight, at least nine, at least ten, at least twelve, at least fifteen, at least twenty, at least twenty five, at least thirty, at least forty, at least fifty samples.

It is preferred that parameter (d) is the particular enzyme. Thus, a particular enzyme is predetermined and the parameters (a) to (c) are selected as reaction temperature, solvent, and solvent concentration and are provided depending on the particular enzyme for the enzymatic reaction.

In this case, the one or more enzyme(s) in step i) comprise or consist of the particular enzyme. Preferably, in step i.a), if present, said enzyme is provided.

It is preferred that parameter (d) is the reaction temperature. Thus, a particular reaction temperature is predetermined and the parameters (a) to (c) are selected as enzyme, solvent, and solvent concentration and are provided depending on the particular reaction temperature for the enzymatic reaction.

In this case, the predetermined temperature range in step i) comprises or consist of the particular reaction temperature. Preferably, the predetermined temperature range in step i.d), if present, comprises or consist of the particular reaction temperature. Preferably, the in step i.d), if present, the ratio of folded to unfolded enzyme is measured at least at said temperature.

It is preferred that parameter (d) is the particular solvent. Thus, a particular solvent is predetermined and the parameters (a) to (c) are selected as enzyme, reaction temperature, and solvent concentration and are provided depending on the particular solvent for the enzymatic reaction.

In this case, the one or more solvent(s) in step i) comprise or consist of the particular solvent. Preferably, in step i.b), if present, said solvent is provided.

It is preferred that parameter (d) is the solvent concentration. Thus, a particular solvent concentration is predetermined and the parameters (a) to (c) are selected as enzyme, reaction temperature, and solvent and are provided depending on the particular solvent concentration for the enzymatic reaction.

In this case, the solvent concentration in step i) comprise or consist of the particular solvent concentration. Preferably, the predetermined solvent concentration in at least one, preferably one, of the at least five samples in step i.e), if present, is the particular solvent concentration.

The term “providing one or more suitable reaction temperature(s), one or more suitable solvent(s), one or more suitable solvent concentration(s), and one or more suitable enzyme(s)” comprises providing the predetermined reaction parameter (d) as well as reaction parameters (a), (b) and (c).

For example, predetermined reaction parameter (d) is a particular enzyme. In this case, one or more suitable reaction temperature(s), one or more suitable solvent(s), and one or more suitable solvent concentration(s)—i.e. reaction parameters (a), (b) and (c)—as well as the enzyme—i.e. predetermined reaction parameter (d)—are provided in step iii), wherein optionally one or more suitable additional enzyme(s) may be provided. The same applies accordingly for a different selection of predetermined reaction parameter (d).

It is preferred that the method according to the invention further comprises the step
iv) conducting an enzymatic reaction based on the
   one or more suitable reaction temperature(s),
   one or more suitable solvent(s),
   one or more suitable solvent concentration(s) of the one or more suitable solvent(s), and
   one or more suitable enzyme(s)
   provided in step iii).

Preferably the method according to the invention further comprising step iv) is a method for conducting an enzymatic reaction comprising the steps of the method according to the invention as described herein and step iv) as described herein.

Preferably, the term “conducting an enzymatic reaction based on” comprises providing the or a substrate of the respective enzyme(s). Further, the term is to be understood such that the enzymatic reaction is conducted with the parameters (a) to (d) provided in step iii), wherein in case for one of parameters (a), (b) or (c) more than one option (e.g. more than one temperature) is provided, the enzymatic reaction is conducted with one of the provided options. Another enzymatic reaction may then be conducted in a parallel or separate run with another one of the more than one options, e.g., with another temperature while keeping the other parameters constant.

Preferably, the enzymatic reaction as described herein is a biotransformation.

Preferably, the term “folded” refers to a polypeptide in (a) conformational state(s) corresponding to that or those occurring in the protein in its biologically active form; its native or undenatured form, as it is present in its natural environment, or after isolation or purification (i.e. before exposure to denaturing conditions); or unique stable intermediates that in subsequent steps may be converted to generate the biologically active species. Preferably, the covalent structure of the folded protein in terms of crosslinking between pairs of cysteine residues in the polypeptide is identical to that of the protein in its biologically active form. Further, the term preferably includes native proteins that may be detectably unfolded to differing extents in their natural environment, and whose folding patterns may change during their natural functioning.

Preferably, the term “unfolded” refers to a polypeptide in conformational states less compact and less-defined than that or those corresponding to the protein in its biologically active, i.e. folded, form. The term thus preferably refers to a biologically inactive polypeptide. Preferably, the term includes a polypeptide, which has lost elements of its secondary and/or tertiary structure that are present in its folded state. Further preferably, the term is not limited to a completely unfolded (e.g. linear, having lost all elements of secondary and tertiary structure) polypeptide but refers to partial or total unfolding. The covalent structure of the unfolded protein in terms of crosslinking between pairs of cysteine residues in the polypeptide may or may not be identical to that of the protein in its biologically active form.

It is preferred that in step i.d) the ratio of folded to unfolded enzyme over a predefined temperature range is measured by a method selected from the group consisting of fluorescence spectroscopy, preferably, nanoDSF; circular dichroism spectroscopy; dynamic light scattering; nuclear magnetic resonance spectroscopy; small-angle scattering, preferably, small angle x-ray scattering; or combinations thereof.

The term “combinations thereof” describes that the method may also comprise more than one of various different methods, i.e. a combination of several methods, which are applied, e.g., subsequently, wherein one or more of the several method(s) may also be applied more than once in the measuring step.

It is further preferred that in step ii), the concentration of the solvent for half-unfolding of the enzyme is determined using a model of two-state unfolding, preferably defined as

$${F}_{350 nm / 330 nm} = \frac{{α}_{F} + {β}_{F} {c}_{s o l v} + ( {α}_{U} + {β}_{U} {c}_{solv} ) exp ( \frac{{m}_{folding}}{R T} ( {c}_{solv} - {c}_{{U}_{50}} ) )}{1 + exp ( \frac{{m}_{folding}}{R T} ( {c}_{solv} - {c}_{U 50} ) )}$$

with
α_{F}, β_{F}, correction factors for linear trends of folded and unfolded signal respectively
α_{U}, β_{U}, to solvent concentration,
cₛₒₗᵥ solvent concentration
m_{folding} proportionality constant of the amount of solvent and the free energy of folding ΔG_{folding},
R universal gas constant
T temperature
c_{U50} solvent concentration at which half of the respective enzyme is folded,
F_{350 nm/330 nm} ratio of folded to unfolded enzyme.

A ratio of folded to unfolded enzyme may be measured by determining the ratio of the fluorescent intensities at 350 to 330 nm. The measurement is based on accessible and non-accessible Tryptophan residues. In the folded state of an enzyme, Tryptophan residues are often buried in the hydrophobic core of a protein, which leads to an fluorescence emission peaking around 330 nm. The residues become exposed during unfolding of the enzyme, which often shifts the fluorescence emission peak toward 350 nm. Thus, the ratio F350 nm/330 nm is often used for determining the ratio of folded to unfolded enzyme.

It is preferred that the model of two-state unfolding is fitted to the measurement data by minimization, wherein, preferably, the minimization includes using the sum-of-least-squares as a cost function.

When using the above definition of the model of two-state unfolding, the parameters α_{F}, β_{F}, α_{U}, β_{U}, m_{folding} and c_{U50} may be used as fitting parameters. For example, it is possible to perform the fitting of the two-state unfolding model using the minimize function of the scipy python package. This may include that the two-state unfolding model as defined above is implemented using the sum-of-least-squares as cost function during the minimization. In particular, the parameters α_{F}, β_{F}, α_{U}, β_{U}, m_{folding} and c_{U50} may be optimized. For the fitting of the model of two-state unfolding, the lower bound may be set, e.g., to 0 for some or all fitting parameters. Furthermore, it is possible to use an upper bound of 100, preferably, only for c_{U50}. The universal gas constant R may be set to 8.314 J mol⁻¹K⁻¹ and T may be set to the respective temperature of the studied system in Kelvin, e.g., the temperature employed during the measurement conducted for obtaining the measurement data.

The present invention further relates to a computer program for providing three reaction parameters (a), (b) and (c) for an enzymatic reaction depending on a reaction parameter (d), wherein the reaction parameters (a), (b), (c) and (d) are selected from the group consisting of a particular enzyme, a reaction temperature, a particular solvent, and the solvent concentration,
wherein each parameter selected from the group consisting of a particular enzyme, a reaction temperature, a solvent, and the solvent concentration is selected as one of the reaction parameters (a), (b), (c) and (d), wherein the computer program including computer-readable instructions, the computer-readable instructions, when executed by a processor of a computer, causing the computer to perform the method according to the invention.

What was said with regard to the method according to the invention applies accordingly to the computer program according to the invention.

The present invention further relates to a non-transitory computer readable data medium storing the computer program according to the invention.

What was said with regard to the method according to the invention or the computer program according to the invention applies accordingly to the non-transitory computer readable data medium according to the invention.

The present invention further relates to a system for providing three reaction parameters (a), (b) and (c) for an enzymatic reaction depending on a predefined reaction parameter (d), wherein the reaction parameters (a), (b), (c) and (d) are selected from the group consisting of a particular enzyme, a reaction temperature, a particular solvent, and the solvent concentration in an aqueous system,
wherein each parameter selected from the group consisting of a particular enzyme, a reaction temperature, a solvent, and the solvent concentration is selected as one of the reaction parameters (a), (b), (c) and (d), the system comprising:
   a providing or receiving unit that is configured for providing or receiving measurement data representing an evolution of the ratio of folded to unfolded enzyme in dependence on a variation of one or more solvent(s) and the solvent concentration(s) of the one or more solvent(s) over a predefined temperature range, for one or more enzyme(s),
   a determination unit that is configured for determining from the evolution of the ratio of folded to unfolded enzyme a concentration of the solvent for half-unfolding of the enzyme for selected temperatures within the predefined temperature range,
   a reaction parameter providing unit that is configured for providing
      one or more suitable reaction temperature(s),
      one or more suitable solvent(s),
      one or more suitable solvent concentration(s), and
      one or more suitable enzyme(s)
   for use in the enzymatic reaction, wherein suitable refers to obtaining a ratio of folded to unfolded enzyme of at least 1:1.

What was said with regard to the method according to the invention or the computer program according to the invention or the non-transitory computer readable data medium according to the invention applies accordingly to the system according to the invention.

In particular, it shall be understood that the aspects described above, and specifically the method of claim 1, the system of claim 16 and the computer program of claim 14, have similar and/or identical preferred embodiments, in particular as defined in the dependent claims.

It shall be further understood that a preferred embodiment of the invention can also be any combination of the dependent claims or above embodiments with the respective independent claim.

These and other aspects of the invention will be apparent from and elucidated with reference to the embodiments described hereinafter.

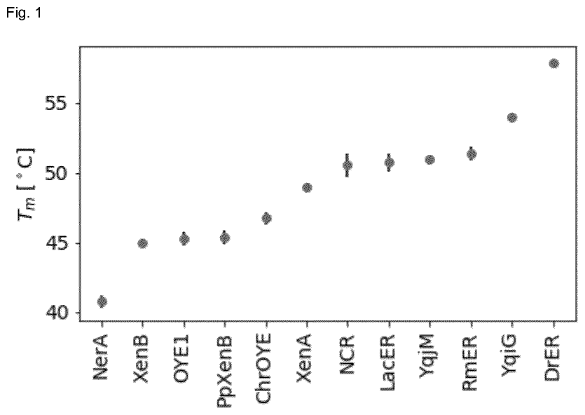

FIG. 1 shows the native melting temperature of the enzymes NerA, XenB, OYE1, PpXenB, ChrOYE1, XenA, NCR, LacER, YqjM, RmER, YqiG and DrER, as described in Example 1.

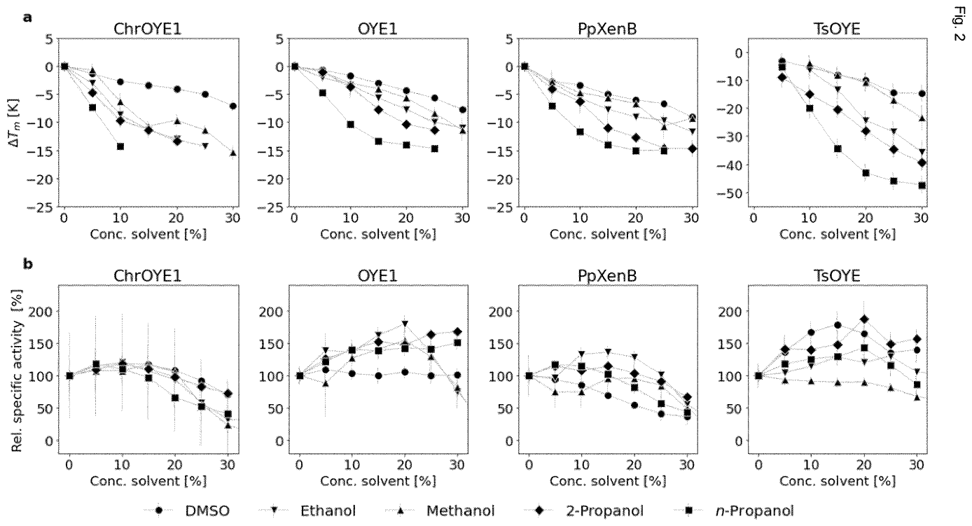

FIG. 2 shows the different (A) melting temperatures and (B) specific enzyme activity in the presence of a solvent, as described in Example 1.

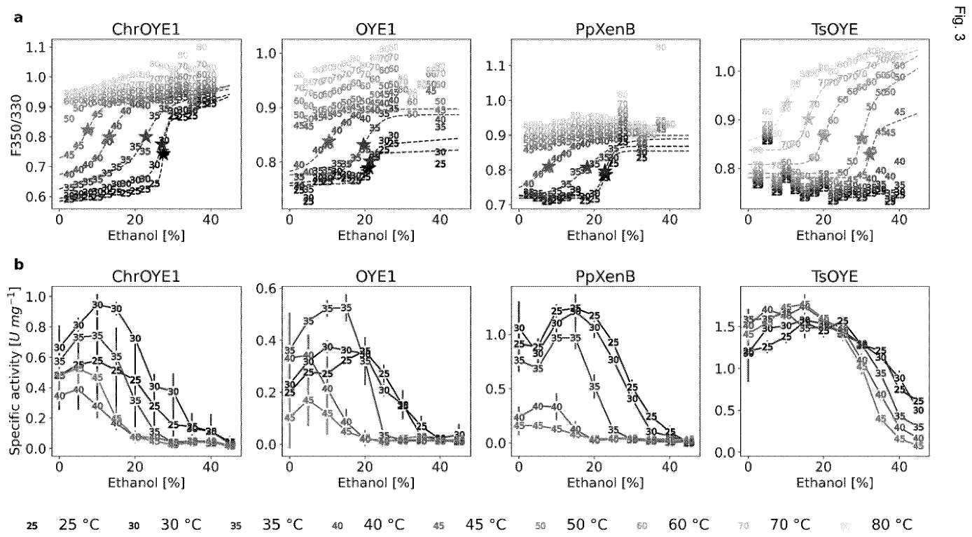

FIG. 3 shows the different (A) proportions of unfolded enzymes and (B) specific enzyme activity for different temperatures and in the presence of ethanol, as described in Example 2.

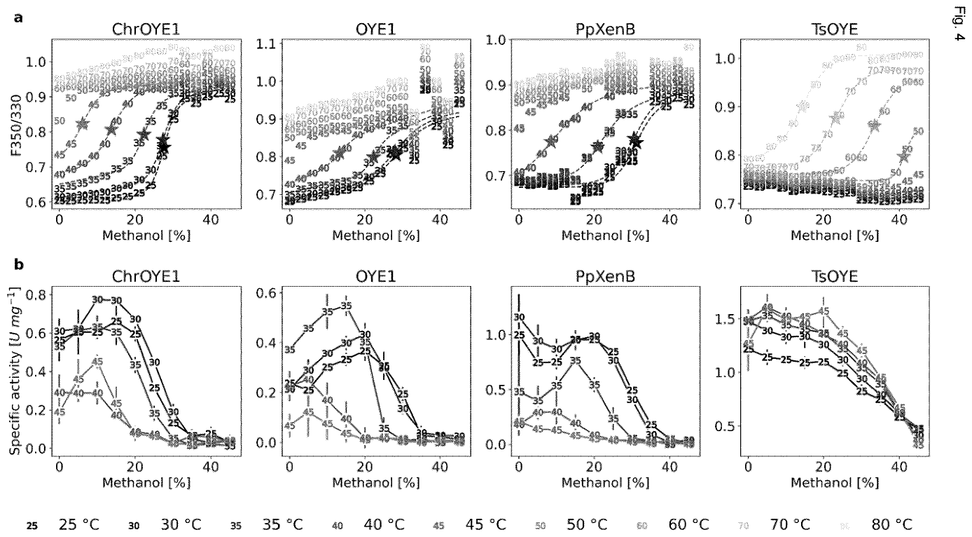

FIG. 4 shows the different (A) proportions of unfolded enzymes and (B) specific enzyme activity for different temperatures and in the presence of methanol, as described in Example 2.

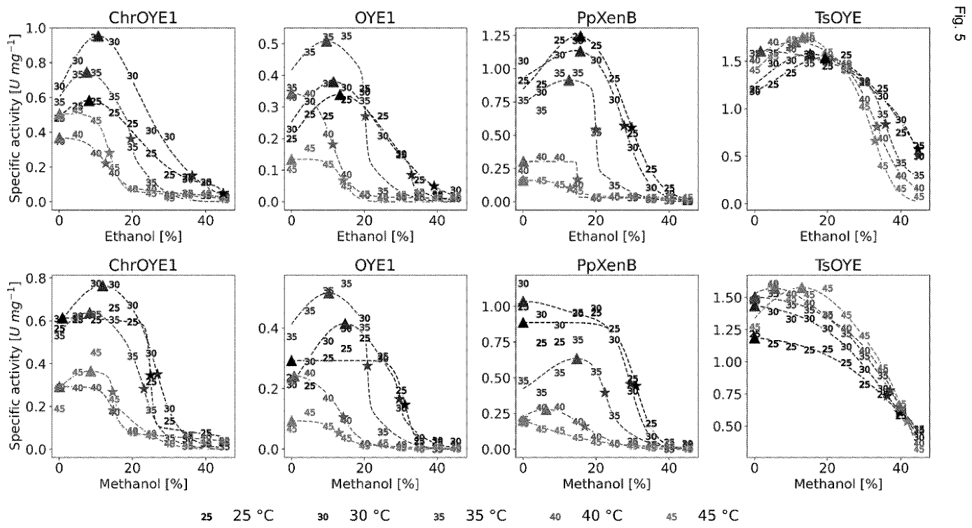

FIG. 5 shows the model for the enzyme activity fitted to the measured data, as described in Example 3.

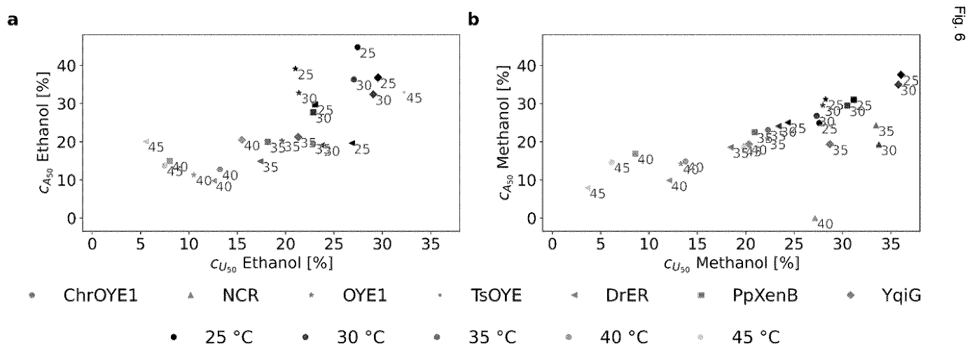

FIG. 6 shows a direct comparison of c_{U50} and c_{A50}, as described in Example 3.

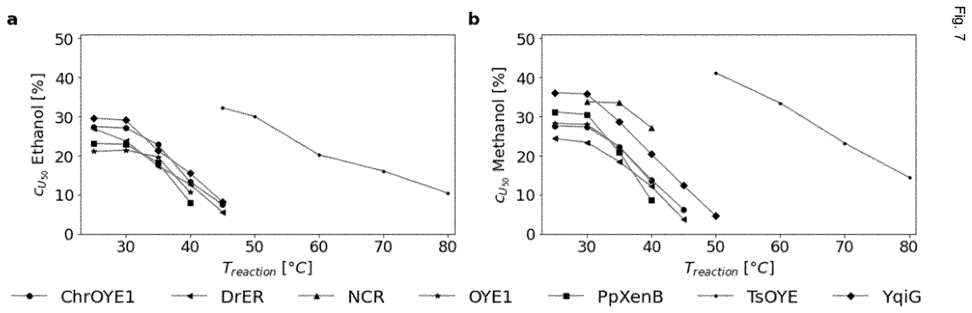

FIG. 7 shows the c_{U50} values for different enzymes depending on the reaction temperature, as described in Example 4.

Further aspects and advantages of the invention result from the subsequent description of preferred examples.

### EXAMPLES

### Example 1: No Correlation Between Melting Temperature and Activity in Solvent-Containing Systems

The melting temperature of 13 ene reductases (NerA, XenB, OYE1, PpXenB, ChrOYE1, XenA, NCR, LacER, YqjM, RmER, YqiG, DrER, and TsOYE) was determined in buffer or buffer with solvent. For this purpose, thermal melting curves of enzymes (1 mg mL-1) in aqueous buffer (50 mM sodium phosphate pH 7.4) supplemented with varying concentrations of solvents (DMSO, ethanol, methanol, n-propanol, 2-propanol) were recorded on a CFX96 Touch Real-Time PCR Detection System (Biorad). The ThermoFMN assay was performed according to Padua, R. A. P., Tomaleri, G. P., Reis, R. A. G., David, J. S., Silva, V. C., Pinheiro, M. P., and Nonato, M. C., “ThermoFMN—A Thermofluor Assay Developed for Ligand-Screening as an Alternative Strategy for Drug Discovery”. J. Braz. Chem. Soc. (2014), 25:1864-1871 in a microplate (MLL9651, Biorad) sealed with Microseal ‘B’ (MSB1001, Biorad). After equilibration to 20° C. the temperature was increased with a gradient of 1° C. min⁻¹ to 90° C. The change of fluorescence upon FMN release was followed using the FAM channel. Melting temperatures were obtained by identifying the inflection point of the recorded curve using python 3. All ThermoFMN experiments were performed in triplicate.

The results are shown in Table 1 and FIG. 1:

TABLE 1

| Enzyme | Melting Temperature [° C.] |
|---|---|
| ChrOYE1 | 46.7 ± 0.5 |
| DrER | 57.9 ± 0.3 |
| LacER | 51.0 ± 0.8 |
| NCR | 50.5 ± 0.8 |
| NerA | 40.7 ± 0.5 |
| OYE1 | 45.3 ± 0.5 |
| PpXenB | 45.3 ± 0.5 |
| RmER | 51.4 ± 0.5 |
| TsOYE | >90 |
| XenA | 49.0 ± 0.0 |
| XenB | 45.0 ± 0.0 |
| YqiG | 54.0 ± 0.0 |
| YqiM | 50.9 ± 0.3 |

In a next step, the melting temperature was measured in the presence of five different organic solvents (DMSO, ethanol, methanol, n-propanol, 2-propanol) at concentrations ranging from 0% to 30% (v/v). From the measured melting temperatures the change of the melting temperature induced by the addition of the solvents was calculated.

It was found that the relative influence of the solvent on the melting temperature across all tested ene reductases is very similar. While DMSO leads to the smallest degree of thermal destabilization across all concentrations, the addition of n-propanol decreases the melting temperature the most. Interestingly, the order of these recorded curves is always the same across all enzymes. DMSO has the smallest effect, then the order is mostly MeOH, EtOH, 2-propanol and finally n-propanol causes the highest reduction of the melting temperature.

The results of the enzymes ChrOYE1, OYE1, PpXenB and TsOYE are shown in FIG. 2A.

As a comparison, the specific enzyme activity was measured at the same solvent condition as the melting temperature as described above.

For this purpose, the reduction of 2-cyclohexene-1-one to cyclohexane-1-one by the respective enzymes was analysed by measuring the consumption of NAD(P)H spectroscopically.

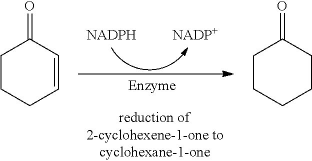 [SMILES: O=C1C=CCCC1 ; O=C1CCCCC1]

The activity of the ene reductases (5-30 μg/mL) was analyzed with 2-cyclohexene-1-one (10 mM, 0.2 mM NADPH/NADH, in 50 mM sodium phosphate buffer pH 7.4 and a reaction volume of 200 μL) as model substrate in a microplate (655101, greiner bio-one). For LacER NADH was used as co-factor, for all other NADPH was used. The reaction was followed by measuring the absorbance of NADPH/NADH at 340 nm on a platereader (SpectraMax M2, Molecular Devices).

The enzyme amount was determined such that the reaction could be followed for 240 s in the linear range. The linear part of the recorded curve was fitted and the slope and the extinction coefficient of NAD(P)H was used to calculate the specific initial activity via Lambert-Beers law. All initial activities are three-fold or five-fold determinations.

TABLE 2

| Order | Compound | c_{stock} | c_{final} | Volume [μL] |
|---|---|---|---|---|
| 1 | Enzyme | | 5-30 μg mL⁻¹ | 6 |
| 2 | ddH₂O | | Add 200 μL | 84-144 |
| 3 | Solvent | | 0-30% | 0-60 |
| 4 | 2-cyclohexene-1-one | 100 mM | 10 mM | 20 |
| 5 | NaPᵢ (10×), pH 7.4 | 500 mM | 50 mM | 16 |
| 6 | NADPH | 2.7 mM | 0.2 mM | 14 |

Surprisingly, the results reveal that the influence of the solvent on the specific activity behaves differently than on the thermal stability. In some cases the addition of smaller amounts of solvents leads to an increased specific activity while higher concentrations of the solvent mostly lead to a loss of activity. Taking both data series into account, no general correlation between the melting temperature and the measured activity could be detected (Pearson correlations coefficient>0.15).

On the contrary, while for the melting temperature a decrease was observed under all tested conditions, the activity was in some cases boosted, in some uninfluenced and in some decreased.

The results of the enzymes ChrOYE1, OYE1, PpXenB and TsOYE are shown in FIG. 2B.

It was thus concluded that the melting temperature or the change of melting temperature by a solvent is not a good measure to draw conclusions on the activity of an enzymatic system.

### Example 2: Solvent Induced Unfolding as Measure for Enzyme Activity

Protein unfolding induced by increasing solvent concentration was measured using nanoDSF, with enzyme concentrations between 0.5 to 2 mg mL⁻¹. The enzymes NerA, XenB, OYE1, PpXenB, ChrOYE1, XenA, NCR, LacER, YqjM, RmER, YqiG and DrER, TsOYE (as described in Example 1) were tested.

Before measuring a sample, it was incubated at the respective condition. 10 μL sample was used in each capillary and heated with a gradient of 2° C. min⁻¹ from 20° C. to 90° C. The change of fluorescence upon unfolding was tracked at 330 nm, 350 nm and the ratio of both.

Following the fluorescent signal of internal tryptophan residues allows tracking structural changes and thus to obtain information on a folded/unfolded state of an enzyme.

The results of the enzymes ChrOYE1, OYE1, PpXenB and TsOYE are shown in FIG. 3A (using ethanol as solvent) and FIG. 4A (using methanol as solvent).

It was found that in general the addition of solvent leads to a transition from the folded to an unfolded state of the respective enzyme. Likewise, increasing the temperature leads to a transition from the folded to an unfolded state of the respective enzyme. Further, by increasing the temperature, the concentration of the solvent needed for unfolding the enzyme was reduced.

Unfolding was analysed for all of the enzymes.

A model for unfolding of an enzyme was used:

$${F}_{350 nm / 330 nm} = \frac{{α}_{F} + {β}_{F} {c}_{s o l v} + ( {α}_{U} + {β}_{U} {c}_{solv} ) exp ( \frac{{m}_{folding}}{R T} ( {c}_{solv} - {c}_{{U}_{50}} ) )}{1 + exp ( \frac{{m}_{folding}}{R T} ( {c}_{solv} - {c}_{{U}_{50}} ) )}$$

with
α_{F}, β_{F}, correction factors for linear trends of folded and unfolded signal respectively
α_{U}, β_{U}, to solvent concentration,
cₛₒₗᵥ solvent concentration,
m_{folding} proportionality constant of the amount of solvent and the free energy of folding ΔG_{folding},
R universal gas constant,
T temperature,
c_{U50} solvent concentration at which half of the respective enzyme is folded.

Fitting this model to the obtained unfolding data provides c_{U50}, the concentration of half unfolding, for each temperature, solvent and solvent concentration. For fitting, the parameters α_{F}, β_{F}, α_{U}, β_{U}, m_{folding} and c_{U50} are optimized. In particular, for example, the fitting of the two-state unfolding model is performed using the minimize function. During the minimization, the sum-of-least-squares as cost function are used. The universal gas constant R may be set to 8.314 J mol⁻¹K⁻¹. The temperature T is set to a predefined constant value, e.g., a value corresponding to a temperature used in a measurement for generating measurement data representing an evolution of the ratio of folded to unfolded enzyme in dependence on a variation of one or more solvent(s) and the solvent concentration(s) of the one or more solvent(s) over a predefined temperature range.

As a comparison, the specific enzyme activity was measured at the same solvent condition as the melting temperature. The specific enzyme activity was measured as described in Example 1, i.e. the reduction of 2-cyclohexene-1-one to cyclohexane-1-one by the respective enzymes was analysed by measuring the consumption of NAD(P)H spectroscopically, however, a larger range of solvent concentrations was measured over different temperatures. The procedure is summarized in Table 3:

TABLE 3

| Order | Compound | c_{stock} | c_{final} | Volume [μL] |
|---|---|---|---|---|
| 1 | Enzyme | | 5-30 μg mL⁻¹ | 6 |
| 2 | ddH₂O | | Add 200 μL | 54-144 |
| 3 | Solvent | | 0-45% | 0-90 |
| 4 | 2-cyclohexene-1-one | 100 mM | 10 mM | 20 |
| 5 | NaPᵢ (10×), pH 7.4 | 500 mM | 50 mM | 16 |
| 6 | NADPH | 2.7 mM | 0.2 mM | 14 |

The results of the enzymes ChrOYE1, OYE1, PpXenB and TsOYE are shown in FIG. 3B (using ethanol as solvent) and FIG. 4B (using methanol as solvent).

Surprisingly, it was found that the solvent concentration influences the enzyme activity and a drop of the activity is observed close to the c_{U50} values for each solvent.

### Example 3: Verifying the Correlation of the Solvent Concentration and the Enzyme Activity

To further examine whether the folded/unfolded state and the c_{U50} are suitable for predicting whether an enzyme may be active or inactive under particular reaction parameters, the above model for folding/unfolding of an enzyme was compared with a model describing the enzyme activity.

For this comparison, the following model describing the enzyme activity was used:

$$Activity = ξ \frac{1}{σ \sqrt{2 π}} exp ( - 0.5 {( \frac{{c}_{solv} - {c}_{{A}_{max}}}{σ} )}^{2} ) + v \frac{exp ( - \frac{{m}_{folding}}{R T} ( {c}_{solv} - {c}_{{A}_{50}} ) )}{1 + exp ( - \frac{{m}_{folding}}{R T} ( {c}_{solv} - {c}_{{A}_{50}} ) )}$$

with
ξ, ν correction factors for scaling,
σ width of maximum,
cₛₒₗᵥ solvent concentration,
c_{A}_{ₘₐₓ} solvent concentration of maximum activity,
m_{folding} proportionality constant of the amount of solvent and the free energy of folding ΔG_{folding},
R universal gas constant,
T temperature,
c_{A}_{₅₀} solvent concentration of steepest drop of activity.

This model describing the enzyme activity was now fitted to the activity data measured as described above (see FIGS. 3B and 4B). As shown in FIG. 5, this model fits the measured data (over the range of reaction temperatures, different solvents and solvent concentrations) very well.

The fitting may be performed using a minimize function. During the minimization, a sum-of-least-squares may be used as cost function. Preferably, in addition, an L2 regularization is added to the cost function. During the fitting, preferably, the parameters ξ, ν, σ, c_{A}_{ₘₐₓ}, m_{folding} and c_{A50} are optimized. Thereby, in general, only the scaling of m_{folding} is needed to avoid biases during the minimization. The lower bounds for ξ, ν, c_{A}_{ₘₐₓ} and c_{A50} may be set to 0. For σ, a lower bound of 10 may be advantageously applied. For c_{A}_{ₘₐₓ} and C_{A50}, upper bounds of 100 may be used, preferably. In all other cases, no bounds may be used. The regularization A may be set to 0.01 for c_{A}_{ₘₐₓ} and to 0.001 for &. In all other cases, A may be set to 0. The universal gas constant R may be set to 8.314 J mol⁻¹K⁻¹ and T may be set to the respective temperature of the studied system in Kelvin, e.g., the temperature applied during the respective measurement.

A direct comparison of all obtained c_{U50} and C_{A50} of all temperature conditions obtained in the measurements as described above is shown in FIG. 6. The direct comparison reveals a correlation between the two values c_{U50} and C_{A50} (Pearson correlation coefficient ethanol: 0.724 and methanol: 0.672). Thus, the solvent concentration of half unfolding is correlated with the solvent concentration of the steepest drop of enzyme activity. This comparison reveals that the model based on c_{U50} instead of the melting temperature (see Example 1) is suitable for predicting whether an enzyme may be active or inactive under particular reaction parameters.

### Example 4: Providing Suitable Reaction Parameters

As demonstrated above, above model can be applied to provide suitable reaction parameters and does not require laborious activity measurements.

For providing such suitable reaction parameters, the solvent-induced unfolding data (i.e. the c_{U50} values) can be provided as described in Example 2 and be plotted with the respective reaction temperature. By analysing the half-unfolding concentration C_{U50} together with the reaction temperature it can be predicted, which solvent concentration and reaction temperature combination will give a folded and which an unfolded enzyme.

Exemplary graphs are shown in FIG. 7.

As shown in FIG. 7, an increased reaction temperature leads to a reduced tolerance of the solvent and an increased amount of solvent leads to a decreased tolerance of temperature.

The obtained curves demonstrate the solvent concentration of half unfolding of an enzyme depending on the temperature. Suitable reaction parameters are all those combinations, which are located at or below the curve of a respective enzyme. Thus, all those combinations are considered to provide a ratio of folded to unfolded enzyme of 1:1 or more, and therefore providing an active enzyme.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like providing or receiving measurement data representing an evolution of the ratio of folded to unfolded enzyme in dependence on a variation of one or more solvent(s) and the solvent concentration(s) of the one or more solvent(s) over a predefined temperature range, and determining from the evolution of the ratio of folded to unfolded enzyme a concentration of the solvent for half-unfolding of the enzyme for selected temperatures within the predefined temperature range, etc. performed by one or several units or devices can be performed by any other number of units or devices. These procedures can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, main-frame computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

1. A computer-implemented method for providing three reaction parameters (a), (b) and (c) for an enzymatic reaction depending on a predefined reaction parameter (d), wherein the reaction parameters (a), (b), (c) and (d) are selected from the group consisting of a particular enzyme, a reaction temperature, a particular solvent, and the solvent concentration in an aqueous system,
wherein each parameter selected from the group consisting of a particular enzyme, a reaction temperature, a solvent, and the solvent concentration is selected as one of the reaction parameters (a), (b), (c) and (d),
the method comprising the steps of:
i) providing or receiving measurement data representing an evolution of the ratio of folded to unfolded enzyme in dependence on a variation of one or more solvent(s) and the solvent concentration(s) of the one or more solvent(s) over a predefined temperature range, for one or more enzyme(s),
ii) determining from the evolution of the ratio of folded to unfolded enzyme a concentration of the solvent for half-unfolding of the enzyme for selected temperatures within the predefined temperature range,
iii) based on the determination of step ii), providing
one or more suitable reaction temperature(s),
one or more suitable solvent(s),
one or more suitable solvent concentration(s), and
one or more suitable enzyme(s)
for use in the enzymatic reaction, wherein suitable refers to obtaining a ratio of folded to unfolded enzyme of at least 1:1.

2. The method according to claim 1, further comprising the step
measuring an evolution of the ratio of folded to unfolded enzyme to obtain measurement data for step i).

3. The method according to claim 1, further comprising the step
iv) conducting an enzymatic reaction based on the
one or more suitable reaction temperature(s),
one or more suitable solvent(s),
one or more suitable solvent concentration(s) of the one or more suitable solvent(s), and
one or more suitable enzyme(s)
provided in step iii).

4. The method according to claim 1, wherein step i) comprises the steps of
i.a) providing one or more enzyme(s),
i.b) providing one or more solvent(s),
i.c) mixing the enzyme(s) provided in step i) with the solvent(s) provided in step ii) to obtain a plurality of samples,
wherein each sample comprises one of the enzyme(s) provided in step i) and one of the solvent(s) provided in step ii) in a predetermined solvent concentration,
wherein for each enzyme provided in step i.a) at least five samples are obtained for each solvent provided in step i.b) and mixed with the enzyme, wherein the predetermined solvent concentration is different between the at least five samples,
i.d) measuring in each of the samples obtained in step i.c) the ratio of folded to unfolded enzyme over a predefined temperature range, and
i.e) generating measurement data representing the measured ratio of folded to unfolded enzyme over a predefined temperature range in each of the samples.

5. The method according to claim 1, wherein parameter (d) is the particular enzyme.

6. The method according to claim 1, wherein parameter (d) is the reaction temperature.

7. The method according to claim 1, wherein parameter (d) is the particular solvent.

8. The method according to claim 1, wherein parameter (d) is the solvent concentration.

9. The method according to claim 1, wherein the enzymatic reaction is a biotransformation.

10. The method according to claim 4, wherein in step i.e) for each enzyme provided in step i.a) at least six samples are obtained for each solvent provided in step i.b) and mixed with the enzyme,
wherein the predetermined solvent concentration is different between the at least six samples.

11. The method according to claim 4, wherein in step i.d) the ratio of folded to unfolded enzyme over a predefined temperature range is measured by a method selected from the group consisting of fluorescence spectroscopy; circular dichroism spectroscopy; dynamic light scattering; nuclear magnetic resonance spectroscopy; small-angle scattering; and combinations thereof.

12. The method according to claim 1, wherein in step ii), the concentration of the solvent for half-unfolding of the enzyme is determined using a model of two-state unfolding.

13. The method according to claim 12, wherein the model of two-state unfolding is fitted to the measurement data by minimization.

14. A computer program for providing three reaction parameters (a), (b) and (c) for an enzymatic reaction depending on a reaction parameter (d), wherein the reaction parameters (a), (b), (c) and (d) are selected from the group consisting of a particular enzyme, a reaction temperature, a particular solvent, and the solvent concentration,
wherein each parameter selected from the group consisting of a particular enzyme, a reaction temperature, a solvent, and the solvent concentration is selected as one of the reaction parameters (a), (b), (c) and (d), wherein the computer program including computer-readable instructions, the computer-readable instructions, when executed by a processor of a computer, causing the computer to perform the method according to claim 1.

15. A non-transitory computer readable data medium storing the computer program of claim 14.

16. A system for providing three reaction parameters (a), (b) and (c) for an enzymatic reaction depending on a predefined reaction parameter (d), wherein the reaction parameters (a), (b), (c) and (d) are selected from the group consisting of a particular enzyme, a reaction temperature, a particular solvent, and the solvent concentration in an aqueous system,
wherein each parameter selected from the group consisting of a particular enzyme, a reaction temperature, a solvent, and the solvent concentration is selected as one of the reaction parameters (a), (b), (c) and (d), the system comprising:
a providing or receiving unit that is configured for providing or receiving measurement data representing an evolution of the ratio of folded to unfolded enzyme in dependence on a variation of one or more solvent(s) and the solvent concentration(s) of the one or more solvent(s) over a predefined temperature range, for one or more enzyme(s),
a determination unit that is configured for determining from the evolution of the ratio of folded to unfolded enzyme a concentration of the solvent for half-unfolding of the enzyme for selected temperatures within the predefined temperature range,
a reaction parameter providing unit that is configured for providing
one or more suitable reaction temperature(s),
one or more suitable solvent(s),
one or more suitable solvent concentration(s), and
one or more suitable enzyme(s)
for use in the enzymatic reaction, wherein suitable refers to obtaining a ratio of folded to unfolded enzyme of at least 1:1.

17. The method according to claim 1, wherein in step i.a) the particular enzyme is provided.

18. The method according to claim 1, wherein in step i.d) the predetermined temperature range comprises or consists of the reaction temperature.

19. The method according to claim 1, wherein in step i.b) the particular solvent is provided.

20. The method according to claim 1, wherein in step ii), the concentration of the solvent for half-unfolding of the enzyme is defined as
$${F}_{350 nm / 330 nm} = \frac{{α}_{F} + {β}_{F} {c}_{s o l v} + ( {α}_{U} + {β}_{U} {c}_{solv} ) exp ( \frac{{m}_{folding}}{R T} ( {c}_{solv} - {c}_{{U}_{50}} ) )}{1 + exp ( \frac{{m}_{folding}}{R T} ( {c}_{solv} - {c}_{{U}_{50}} ) )}$$
with
α_{F}, β_{F}, correction factors for linear trends of folded and unfolded signal respectively
α_{U}, β_{U}, to solvent concentration,
cₛₒₗᵥ solvent concentration
m_{folding} proportionality constant of the amount of solvent and the free energy of folding ΔG_{folding},
R universal gas constant,
T temperature,
c_{U50} solvent concentration at which half of the respective enzyme is folded, and
F_{350 nm/330 nm} ratio of folded to unfolded enzyme.
